(19) United States
(12) Patent Application Publication
(10) Pub. No.: US 2025/0339045 A1
(43) Pub. Date: Nov. 6, 2025

(54) Photoplethysmography Based Atrial Fibrillation Detection System and Method

(71) Applicant: Academia Sinica, Taipei (TW)

(72) Inventors: Fu-Liang YANG, Hsinchu City (TW); Justin CHU, Taoyuan City (TW); Wen-Tse YANG, New Taipei City (TW)

(21) Appl. No.: 18/862,547

(22) PCT Filed: May 18, 2023
(86) PCT No.: PCT/US2023/022635

§ 371 Date: Nov. 3, 2024

Related U.S. Application Data
(60) Provisional application No. 63/345,080, filed on May 24, 2022.

(51) Int. Cl. A61B 5/024; A61B 5/00

(52) U.S. Cl. CPC A61B 5/02416; A61B 5/7282

(57) ABSTRACT

The present invention provides an atrial fibrillation detection system and method based upon irregularity in inter-pulse duration or interval of a subject's cardiovascular signal. Specifically, the present invention determines existence of atrial fibrillation based upon irregularity in percentage difference in duration or interval of consecutive pulses of a subject's cardiovascular signal. In addition, the present invention applies analysis of flux-interval plots of pulse interval and pulse normalized amplitude to screen out false positives and displays the flux-interval plots to the subject to provide transparency to the subject.

### TECHNICAL FIELD OF THE INVENTION

The present invention relates to detection of atrial fibrillation system and method. More specifically, the present invention relates to detection of atrial fibrillation system and method based on irregularity in inter-pulse (consecutive pulse) duration or interval.

### BACKGROUND OF THE INVENTION

Atrial fibrillation (AFib) is the main factor of cardioembolic stroke and is associated with a 3.7-fold increase in all-caused death [1]. AFib happens when the atrium depolarizes fast and irregularly, which leads to contractile dysfunction. However, adequate treatments are hindered from those patients with AFib due to as many as 50˜87% of them being initially asymptomatic [2]. Thus, accurate and convenient automated AFib detection methods have always been a popular research topic for its demands and challenges.

The golden standard of AFib detections is conducted through analyzing 12-lead electrocardiogram (ECG) signal which is inconvenient for large-scale screening programs and not suitable for continuous or long-term monitoring. Just like ECG, other cardiovascular signals such as photoplethysmogram (PPG) signal also originates from the cardiac cycle which inherits the ability to extract the same variability features but is more circumstantial as it is the measurement of blood flow volume difference in the capillary caused by each heartbeat. A study on the extracted features between ECG and PPG had been conducted to confirm the viability of using PPG to replace ECG for heart rate variability (HRV) features [3]. PPG signals also reflect one's hemodynamic characteristics that contain full information of cardiac activity, cardiovascular condition, sympathetic and parasympathetic nervous system interaction, and hemoglobin level from a peripheral site [4-6]. These characteristics could shed new light on different AFib detection methods by revealing new information, namely the change in every heartbeat's stroke volume, that wasn't accessible through ECG. However previous studies are fixated on only using interval related features shared by both ECG and PPG.

PPG sensors are more affordable, easier to use, and already commonly implemented on various wearable devices making it a potentially convenient alternative for AFib detection [7]. A common application is to combine a PPG device with a mobile phone by either connecting the phone to a device with PPG sensors or utilizing the smartphone's camera as the PPG sensor. A demonstration of use case scenario with smartphone using our methodology is depicted in FIG. 1. The figure shows a concept of a mobile phone app presenting the users with information regarding the detection result of the analyzed PPG signal.

A detailed review of previous studies had been conducted by Tania Pereira et al. on different approaches of PPG-based AFib detection [8]. In their paper, previous works are split into three groups by the approaches they use, which are statistical analysis approaches, machine learning approaches, and deep learning approaches. As Pereira et al. mentioned, previous PPG-based AFib detection methods suffer from many shortcomings and face many different challenges on different aspects, such as having difficulty in distinguishing AFib from other AFib-like cardiac arrhythmia, and over-complicated models for users to interpret the results.

In general, statistical analysis approaches extract RR interval (R peak to R peak) series features and spectral entropy and then try to find the best threshold values with ROC curves [9-13]. Simple statistical analysis methods are robust and intuitive but can be less effective compared to the more advanced machine learning and deep learning methods.

Due to the randomness of atrial contraction for AFib in combination with significant variability on inter-personal differences, the PPG signals can present in many different waveforms. Due to these immense differences, for machine learning methods to cover all the bases might require an immensely large amount of training data which can be very hard to come by. While machine learning methods offer more effective and optimized algorithms, much like the statistical analysis methods, their performance is still bound by the limitation of the quality of the features that were available to the model.

To overcome this limitation, researchers turn to deep-learning approaches with automatic feature extraction such as convolution neural network (CNN). Convolution neural networks are commonly used in solving image-related problems for their ability to extract important and representative patterns as features automatically through different filters from the

data input [14]. Kwon et al. applied CNN models and mentioned that previous algorithms were mostly based on RR interval series and HRV related features and there has been very little discussion on PPG signal amplitude [15]. Deep learning approach with CNN layers may allow the model to gain amplitude information, but due to the nature of being a black-box algorithm, it is hard to interpret or confirm how and whether the model actually uses the amplitude information.

Methods proposed in previous works may achieve promising performance but the explaicability and transparency of the detection model were not very reassuring from a typical user's point-of-view. Based on the work by Pereira et al., we further organized the previous studies into different groups by where their feature arise from each study used in Table 1 to categorized different approaches and show how underutilized the pulse amplitude information is.

There is a need for an atrial fibrillation detection system and method that accurately distinguish AFib from other cardiac arrhythmia and normal sinus rhythm (NSR) by analyzing one's cardiovascular pulse changes which acts as a surrogate of beat-to-beat stroke volume variation. There is also a need for an atrial fibrillation detection system that provides a visual characterization of the physiological basis for every prediction result. This would help inform the users of the basis of the model's decision to bolster user confidence and allow for a second opinion to double-check the result as previous works were able to give users a concise result but users may find it obscure and out of touch.

### BRIEF DESCRIPTION OF THE DRAWINGS

FIG. 1 illustrates an embodiment of the atrial fibrillation detection system 10 of the present invention with flux-interval plots (tᵢ, Hᵢ) 260 and (tᵢ₋1, Hᵢ) 262 displayed on a controller 200 such as a smart phone for visual assessment by a subject.

FIG. 2 depicts in detail an embodiment of the atrial fibrillation detection system 10 of the present invention.

FIG. 3 illustrates in detail the controller 200 of the atrial fibrillation detection system 10 of the present invention.

FIG. 4 depicts an embodiment of H-index-inspired interval irregularity index (III) calculation. FIG. 4A shows the % of samples Pⱼ satisfying a particular threshold value Tⱼ. FIG. 4B shows the quadratic mean of P&T of equation (1) across different thresholds Tⱼ and where the minimum value is located with the result of III being 18.0.

FIG. 5 illustrates the transformation of PPG signals to flux-interval plot of Hᵢ vs tᵢ.

FIG. 6 illustrates examples of orthogonal regression line on main cluster of PVC data (FIG. 6A) and AFib data (FIG. 6B).

FIG. 7 depicts irregularity distribution of different types of heart beat rhythm and automatic detection process of separating AFib from different arrhythmic rhythm with irregularity and RMSE of orthogonal regression on the main cluster from (ti, Hi) relation. FIG. 7A maps the distribution with interval irregularity index as the x-axis and amplitude irregularity index as the y-axis. FIG. 7B maps the distribution using a bar graph showing specifically data for NSR and PAC/PVC. FIG. 7C maps the distribution of AF, PAC/PVC, and other, using regression RMSE of main cluster at 0.06 as the boundary. FIG. 7D shows the results of AF/Non-AF prediction against actual AF/Non-AF.

FIG. 8 illustrates an embodiment of the atrial fibrillation detection method 1000 of the present invention.

FIG. 9 illustrates typical pattern of flux-interval plots 260, 262 for different cardiac rhythms. Blue dots represent the main cluster for each sample in the plot. FIG. 9A illustrates flux-interval plots in the configuration of Hᵢ vs tᵢ, while FIG. 9B illustrates flux-interval plots in the configuration of Hᵢ vs tᵢ₋₁.

FIG. 10 illustrates two false-positive cases of premature contractions (non-AFib) classified as AFib, wherein interval irregularity is 24.7 in FIG. 10A and 24.6 in FIG. 10B. In both cases, frequent PACs were encountered but these PACs came with different coupling intervals if measured carefully (FIGS. 10A and 10B). In addition to the PAC attacked in case of FIG. 10A, a short-run atrial tachycardia (AT) attacked around time mark 39 seconds.

FIG. 11 illustrates two cases of non-AFib (ECG labeled as others, Atrioventricular block) correctly detected, wherein interval irregularity is 41.9 in FIG. 11A and 55.2 in FIG. 11B. In both cases, their irregularity indexes are very high but their regression RMSE on main cluster is less than the threshold of 0.06. Thus, they are automatically detected as non-AFib. Their flux interval configurations are similar to PAC/PVC but far from AFib.

### DETAILED DESCRIPTION OF THE INVENTION

As used in this specification and in claims which follow, the singular forms “a”, “an,” and “the” include plural referents unless the context clearly indicates otherwise. Thus, for example, reference to “an ingredient” includes mixtures of ingredients; reference to “an active pharmaceutical agent” includes more than one active pharmaceutical agent, and the like. As used herein, the term “about” as a modifier to a quantity is intended to mean +or −10% or +or −5% inclusive of the quantity being modified.

The compositions of the present invention can comprise, consist of, or consist essentially of the essential elements and limitations of the invention described herein, as well as any of the additional or optional ingredients, components, or limitations described herein.

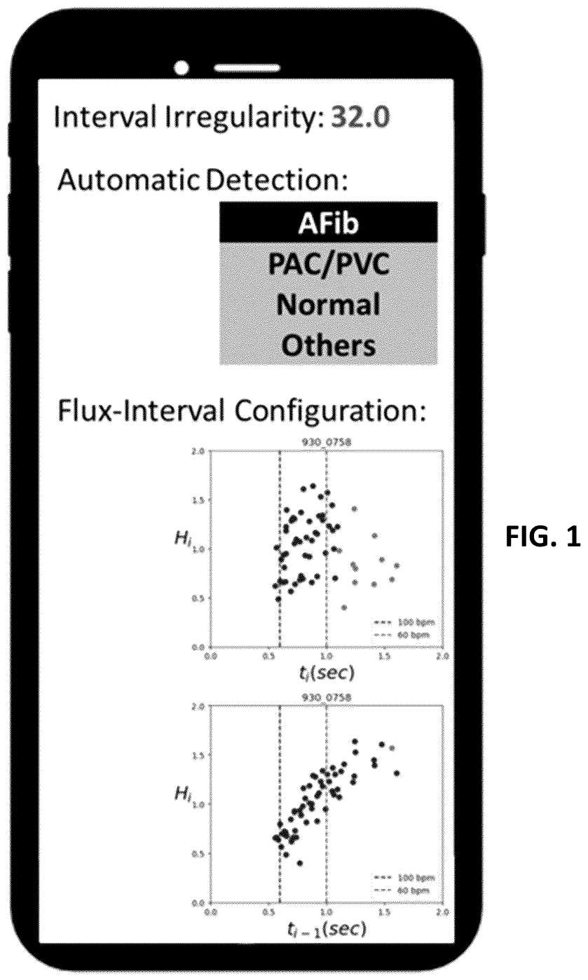

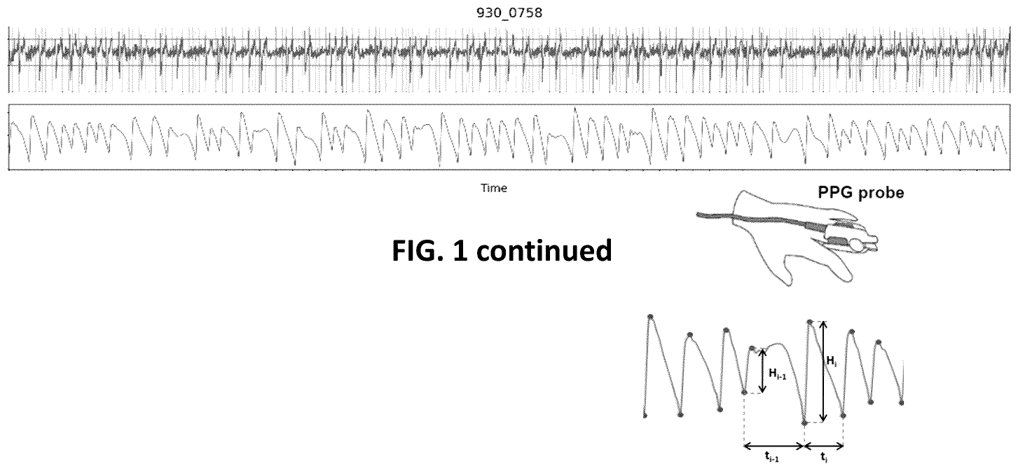

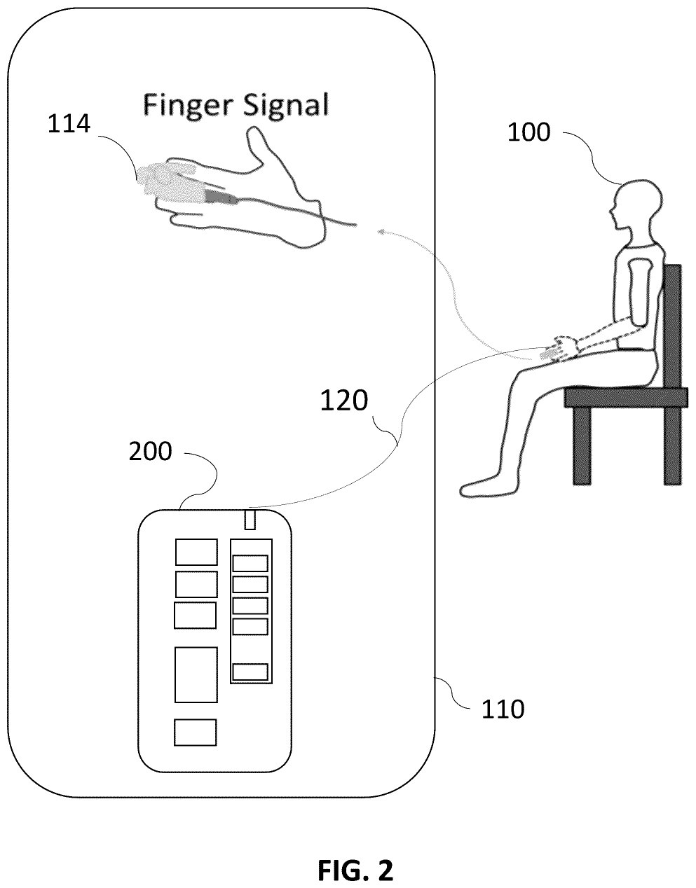

The present invention provides an atrial fibrillation detection system 10, an embodiment of which is depicted in general in FIG. 1 and in more detail in FIG. 2. As shown in FIG. 2, in an embodiment, the atrial fibrillation detection system 10 of the present invention comprises a cardiovascular signal sensor 114, a connector 120 and a controller 200. In an embodiment, the signal sensor 114 is configured to output and read signals emanating from the subject 100. The signal may comprise optical, mechanical, electrical, acoustic, thermal signal or a combination thereof. In one embodiment, the signal sensor 114 comprises any commercially available photoplethysmogram (PPG) signal sensor capable of communicating with the controller 200. In an embodiment, the signal sensor 114 comprises a signal reader112 configured to read signals emanating from the finger of the subject 100. In an embodiment, the signal sensor 114 may further comprise a signal emitter 113 configured to output signals capable of passing through the body of the subject 100, then emanate out from the subject 100 to be read by the signal reader 112.

In an embodiment, connector 120 is configured to allow communication between the signal sensor 114 and the controller 200. In an embodiment, the connector 120 may transmit the signal segment 101 read by the signal sensor 114 to the controller 200 as well as to the signal sensor 114 to emit and/or read signals. In an embodiment, the signal segment 101 may comprise signal of about 30 seconds to about 2 minutes, such as about 30 seconds, about 40 seconds, about 50 seconds, about 1 minute, about 1.2 minutes, about 1.4 minutes, about 1.6 minutes, about 1.8 minutes or about 2 minutes including any numbers and number ranges falling within these values. In one embodiment, the connector 120 may be a physical wire, in another embodiment, the connector 120 may comprise a wireless connection such as those using Wi-Fi or Bluetooth technology.

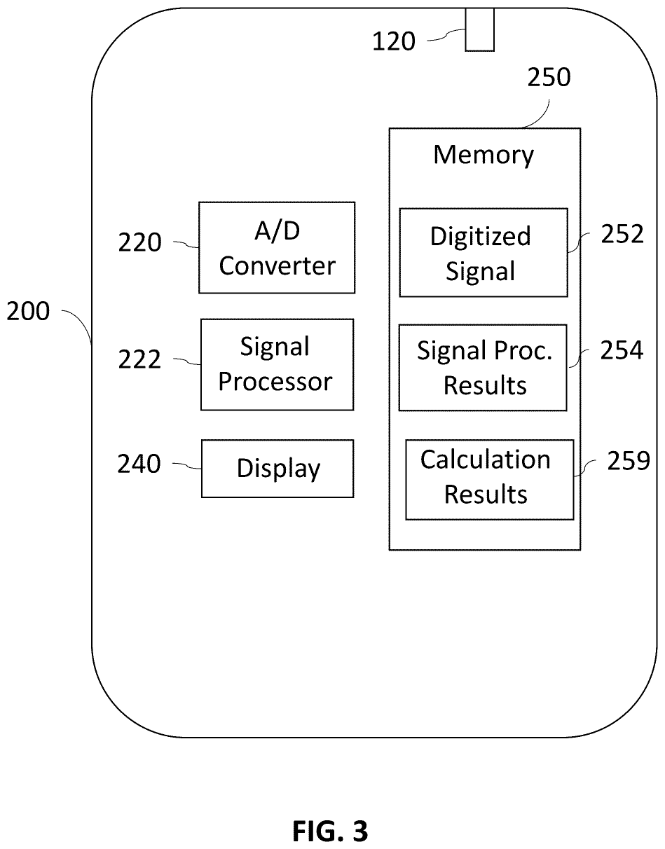

FIG. 3 illustrates an embodiment of the controller 200 in further detail. As shown in FIG. 2, in an embodiment, the controller 200 comprises an analogue to digital converter (A/D converter) 220, processor 222, display 240 and memory 250. In an embodiment, memory 250 comprises digitized signal segment 252, signal processing results 254 and calculation results 259.

In an embodiment, the A/D converter 220 is configured to digitize the analogue signal segment 101 transmitted to the controller 200 into digitized signal segment 252, which may be stored in memory 250 for further processing. In an embodiment, the processor 222 is configured to communicate with and control the signal reader 112, signal emitter 113 of the signal sensor 114 so that the user 100 is able to control the sensor 114 to emit signals and capture signals using user interactive display 240.

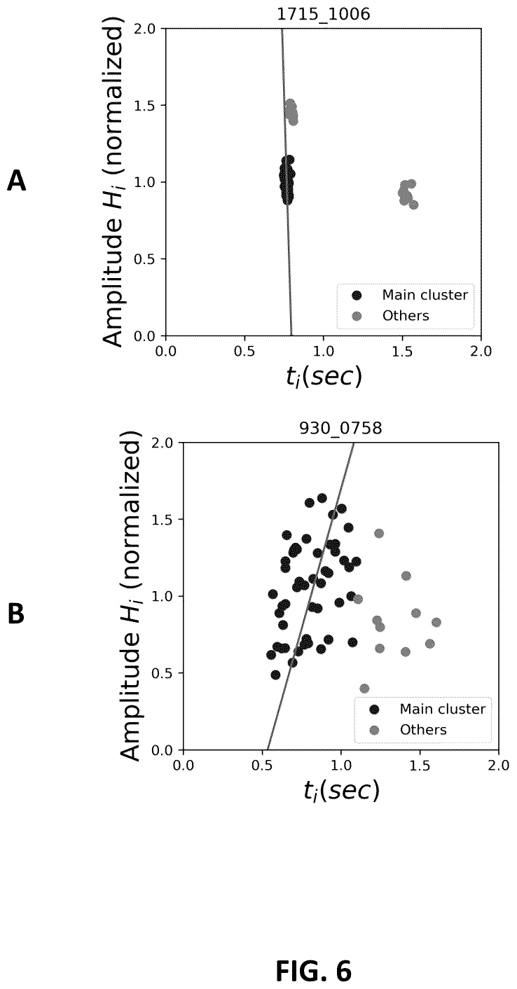

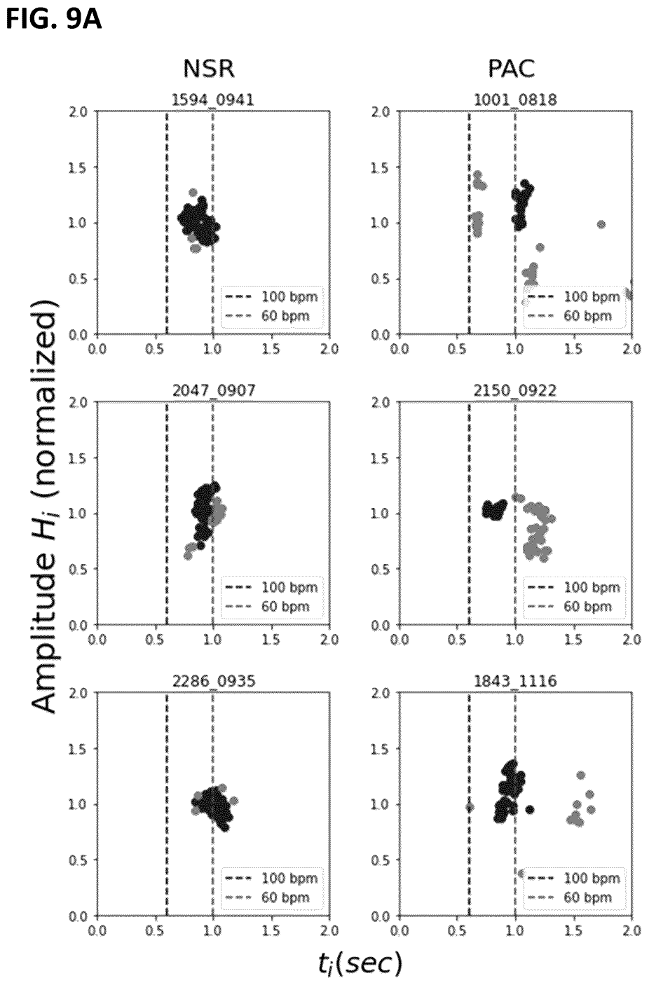

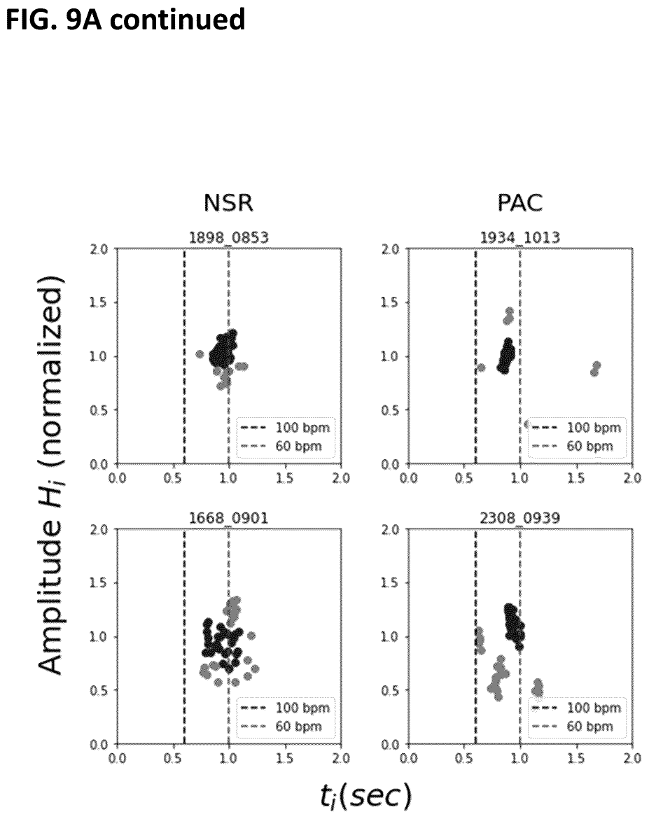

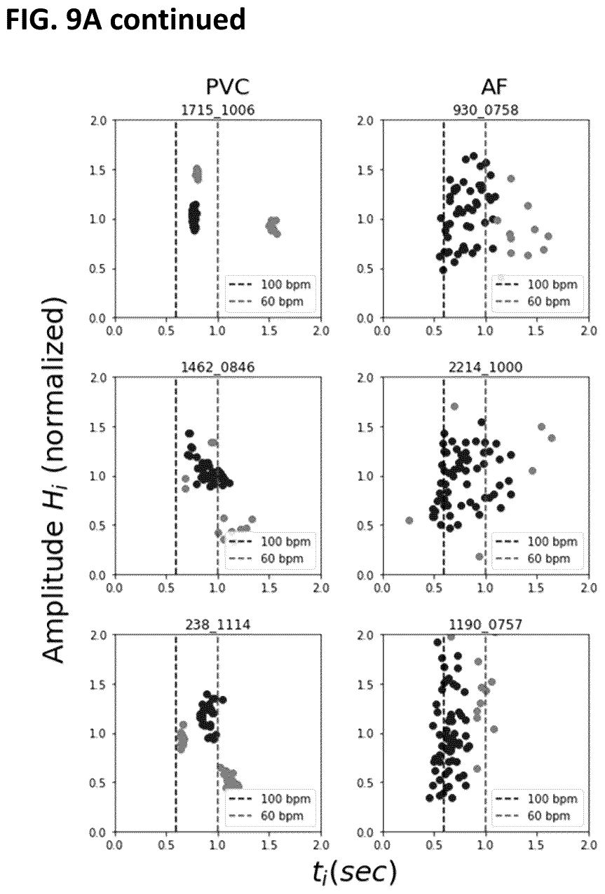

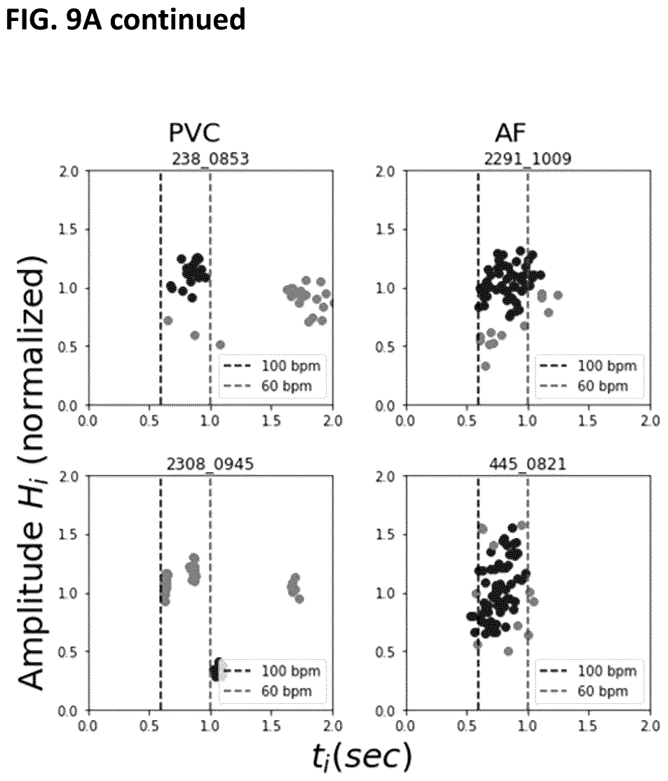

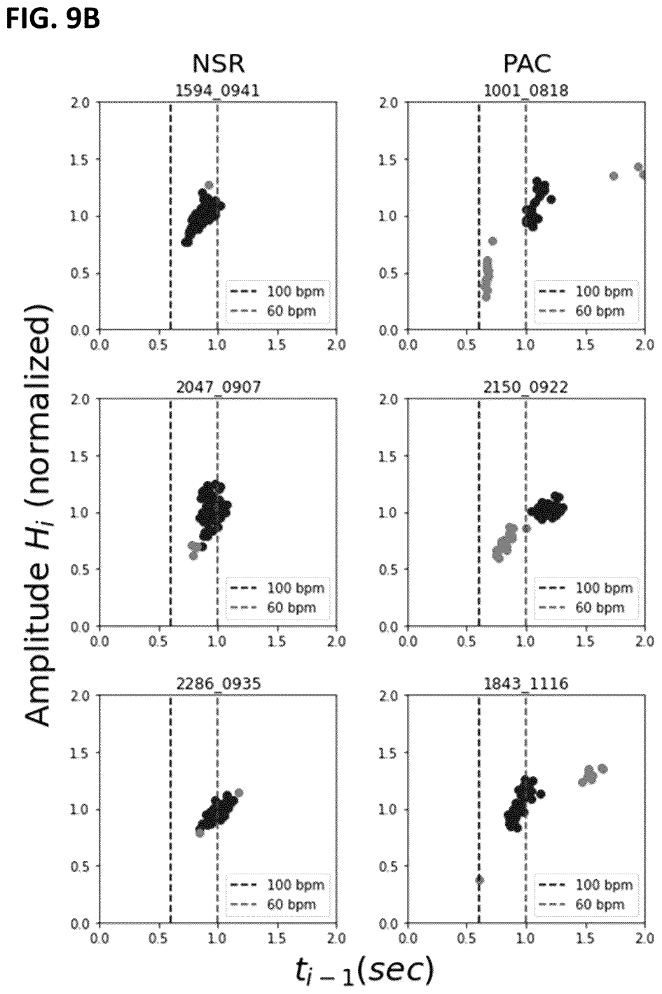

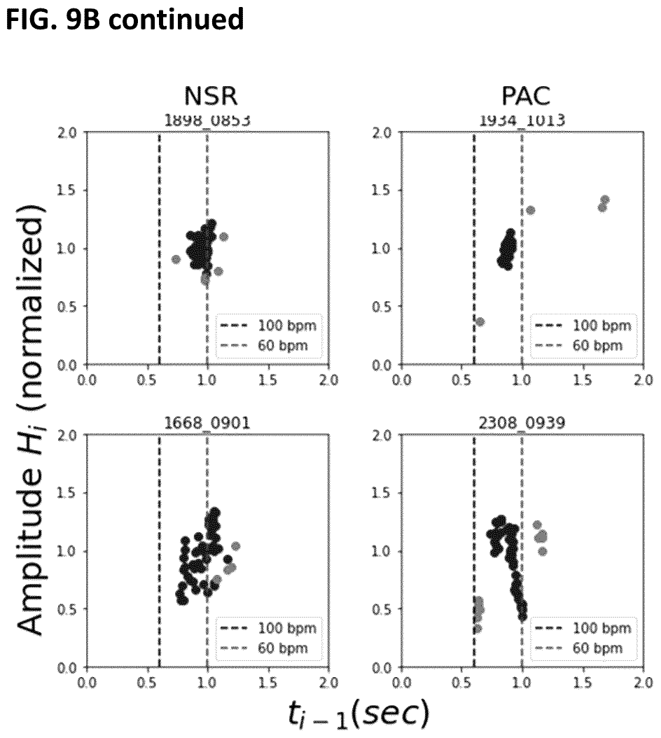

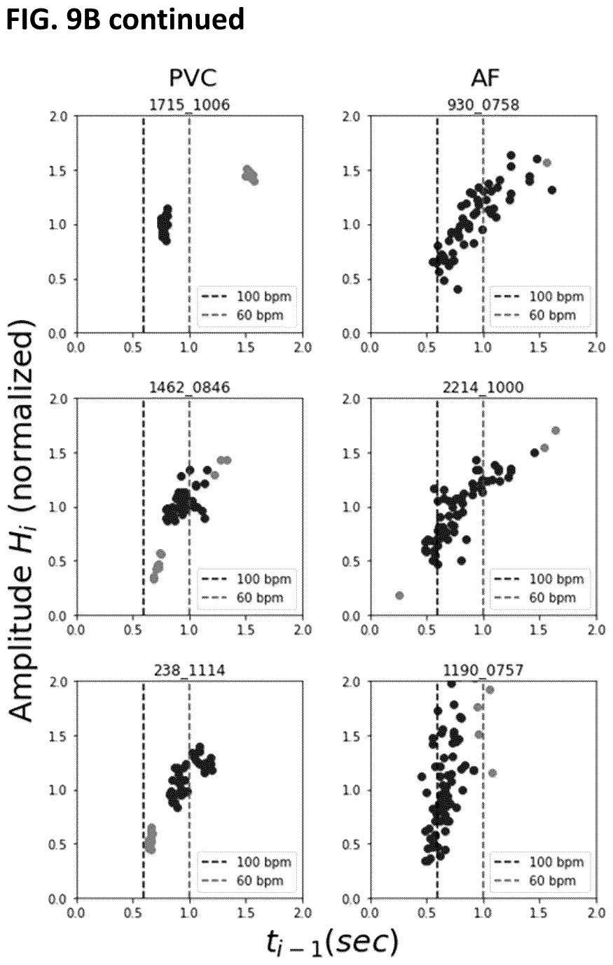

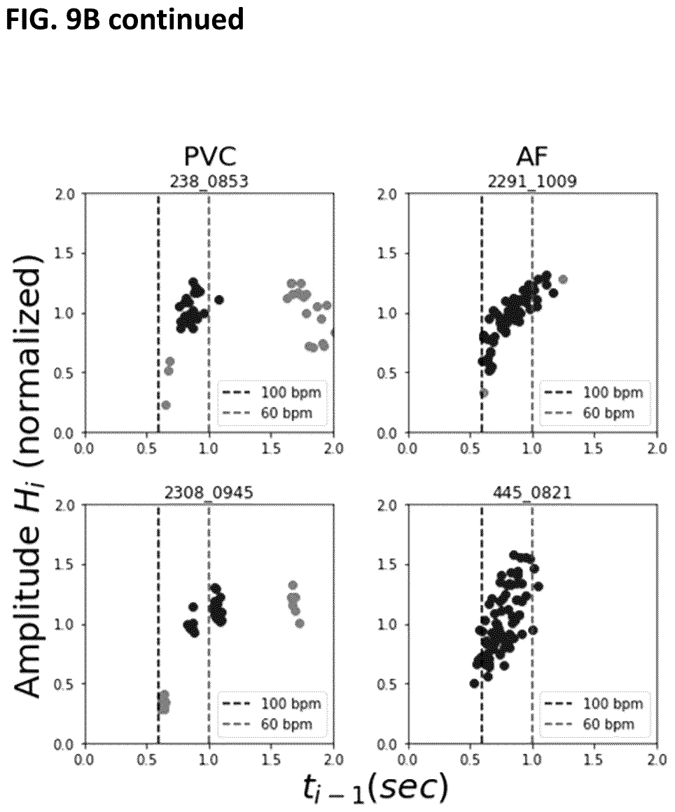

In addition, in an embodiment, the processor 222 is configured to process the digitized signal segment 252. In an embodiment, the processor 222 is configured to detect peaks and valleys of pulses of the digitized signal segment 252 to identify individual pulses within the digitized signal segment 252. In an embodiment, the processor 222 is configured to process the digitized signal segment 252 into individual pulses 254 indexed by i. In an embodiment, the processor 222 is configured to calculate and associate each pulse 254 with pulse interval or duration ti 256 and normalized pulse amplitude Hi 258 wherein the normalized pulse amplitude Hi 258 is the pulse peak amplitude divided by its average. The sets of (tᵢ, Hᵢ) and (tᵢ₋₁, Hᵢ) may be visualized for the subject 100 as flux-interval plots 260 and 262 respectively on display 240 as illustrated in FIG. 6. From the flux-interval plots 260, 262, it is possible to observe that normal sinus rhythm (NSR), premature atrial contraction (PAC), premature ventricle contraction (PVC), and atrial fibrillation (AFib) samples display distinctively different patterns. Flux-interval plots 260, 262 allow the subject 100 to visually assess his or her changes in cardio-output over time and identify different cardiac rhythms with their distinctive patterns much like identifying AFib from ECG using only a few criteria. The system of the present invention 10 would allow any well-informed subject 100 to differentiate normal and abnormal rhythms based on unique patterns with minimum training as discussed in further details below in connection with FIGS. 9-11. Therefore, in an embodiment, the signal processor 222 is configured to construct flux-interval plot (tᵢ, Hᵢ) 260 and (tᵢ₋₁, Hᵢ) 262 and display the plots 260, 262 on display 240.

In an embodiment, the processor 222 is further configured to analyze the digitized signal segment 252 and pulses 254 to determine whether a digitized signal segment 252 contains atrial fibrillation. In an embodiment, the processor 222 is configured to process the digitized signal segments 252 and its individual pulses 254 to calculate values such as interval irregularity index (III) 300 and Regression RMSE of Main Cluster 400, including application of density-based spatial clustering of applications with noise (DBSCAN) clustering to the flux-interval plot 260 to identify or define the main cluster, that can be very useful in determining whether a particular signal segment 252 contains atrial fibrillation pulses.

In an embodiment, the III 300 is defined as:

$$III = min {\{ \sqrt{\frac{{T}_{i}^{2} + {P}_{i}^{2}}{2}} \}}_{i = 1}^{50} \qquad ( 1 )$$

wherein Tᵢ 257 is the threshold percentage difference of consecutive pulse interval tᵢ 256 and Pᵢ 255 is the proportion of pulses 254 of a signal segment 252 that satisfies the interval threshold Tᵢ 257.

In an embodiment, the III index 300 is a H-index inspired index designed to represent irregularity of each signal segment 252 and can be used to assist in determining if the signal segment 252 contains atrial fibrillation (AFib) pulses. The III index 300 is calculated by finding the smallest quadratic mean (root mean square) of the thresholds of percentage difference of consecutive pulse interval (T) 257 and the proportion of pulses 254 (P) 255 whose percentage difference of consecutive pulse interval is equal to or greater than said interval threshold T257 as summarized in Equation (1). While previous studies often use RR time interval features in an absolute time difference of millisecond, we opt to use the relative difference in percentage when it comes to consecutive pulse duration or interval difference. Specifically, using percentage difference rather than raw value of consecutive pulse duration or interval difference allows the present invention to also take account of the heart rate, resulting in superior atrial fibrillation analysis. For instance, a 10 millisecond change in pulse between a person with a heart rate of 60 beats/minute and another person with a heart rate of 80 beats/minute do present a significant difference for signal analysis. Thus, the present invention uses the percentage difference rather than raw value difference of inter-pulse intervals tᵢ. The process of finding the III index 300 is illustrated graphically in FIG. 3.

In an embodiment, the Regression RMSE of Main Cluster 400 comprises root mean squared error of the main cluster within plots of (tᵢ, Hᵢ) 260 for a particular signal segment 252 wherein tᵢ 256 is the interval or duration of the i^{th} pulse, Hᵢ 258 is the normalized amplitude of the i^{th} pulse, and the main cluster refers to main cluster of a flux interval plot (tᵢ, Hᵢ) 260 as illustrated in FIG. 6 (the cluster with the most data). In an embodiment, the main cluster of a flux interval plot (tᵢ, Hᵢ) 260 is identified by using DBSCAN. In other embodiments, identification of the main cluster may comprise K-means, Gaussian mixture model algorithm, Mean shift etc. . . . Orthogonal regression is then applied to the main cluster of (tᵢ, Hᵢ) 260 sets and the residual errors were recorded in the form of root-mean-squared error (RMSE). As examples shown in FIG. 6, we expect the fitted regression line of main cluster would result in a smaller RMSE on premature contractions as they tend to have tight clusters as opposed to the more scattered distribution of AFib on the flux-interval plot. We intend to use the Regression RMSE of Main Cluster 400 as a simple index to represent the degree of dispersion of each signal 252. The clustering result would reflect the types of pulses within the signal segment 252 based on each pulse's location in the flux-interval plot. Types of pulses may comprise atrial fibrillation, NSR, premature atrial contraction (PAC), premature ventricle contraction (PVC), etc. . . .

Previous studies such as analysis of cardiac arrhythmia by Pfeiffer et. Al. had indicated a strong correlation between (tᵢ₋₁, Hᵢ), but the reasoning behind this correlation was not

clearly explained [33]. Here we try to explain the hemodynamic cause of (tᵢ₋₁, Hᵢ) correlation in detail as below, and our algorithm would also use cardiac electrophysiology to enhance the discrimination power.

Hemodynamic model and (tᵢ₋₁, Hᵢ)

A person's stroke volume is determined by how much blood is ejected during contraction. As shown in Equation (2) below, there are three factors affecting one's stroke volume (or flux), namely preload, contractility, and afterload. During the diastole of the heart, the blood accumulates in the ventricle before the ventricular contraction, and the end-diastolic pressure is so-called preload. The normal diastole starts with rapid filling due to passive ventricular suction and follows with active filling by atrial contraction. While ejecting the blood from the heart, it has to overcome the systematic arterial pressure that is pushing back against the aortic valve which is referred to as afterload.

$$Stroke volume = f ( Preload , Contractility , Afterload ) \qquad ( 2 )$$

For simplicity, it is assumed that for each person within each one-minute measurement, their contractility and afterload should remain largely the same, thus these values are treated as constant here. Therefore, we substitute the Contractility and Afterload with a Constant to transform the model function into Equation (3).

$$Stroke volume = {f}_{p} ( Preload , Constant ) \qquad ( 3 )$$

During cardiac cycles, the preload period for pulse i is represented by the preceding pulse's interval tᵢ₋₁. In the early passive diastolic phase, the atrium works as a reservoir of blood and the filling volume is related to the ventricular suction pressure and the filling time. Since the preceding pulse interval (tᵢ₋₁) would largely affect the filling time and the flow rate is in proportion to ventricular suction pressure, the integral of these 2 items may represent for the preload, which like an hourglass between heart contractions, we formulated Equation (4).

$$Stroke volume = {f}_{p} ( flow rate ( s ) \times {t}_{i - 1} ) , active filling \qquad ( 4 )$$

Since the atrial mechanical function is impaired in AFib, the active diastolic filling is negligible in AFib. This simplified the model into Equation (5) and allows us to focus on how the preload period affects one's stroke volume.

$$Stroke volume = {f}_{p} ( flow rate ( s ) \times {t}_{i - 1} ) \qquad ( 5 )$$

PPG amplitude is in proportion to stroke volume but their relationship has yet to be properly modeled. The PPG signal amplitude is correlated to the amount of blood flow but is hard to model due to many other different variables such as systematic vascular resistance. These various variables can result in inter-and intra-personal differences when comparing. Here, we assume that within each one-minute PPG measurement the intra-personal difference on variables other than stroke volume remains largely the same thus can be neglected. Based on this assumption we can interchange the stroke volume with PPG amplitude and result in Equation (6).

$$Amplitude ( {H}_{i} ) = flow rate ( s ) \times {t}_{i - 1} \qquad ( 6 )$$

This explains why flux-interval plot of Hᵢ vs tᵢ₋₁ would present with a positive slope especially in patients with AFib.

Electrophysiology model and (tᵢ, Hᵢ)

The AFib is well-known as chaotic heart rhythm, and previous studies aimed to evaluate the randomness of AFib had found the auto-correlation between each RR intervals was low [34]. However, this relationship would not be random in sinus rhythm, PAC, or PVC.

The coupling interval, namely the RR-interval preceding the premature beats (tᵢ₋₁), is traditionally believed to be constant in a stable sinus cycle length [35]. In addition, the ECG morphology of a premature beat has a fixed relationship with its coupling interval. It is because the firing of a PAC or PVC is originated from the same piece of the myocardium with the same mechanism. Although various kinds of premature beats may be present in a patient, a dominant morphology with its fixed coupling interval would be observed more frequently.

The relationship of returning cycles, namely the RR-interval following the premature beats (tᵢ), is also not random [36]. When a PAC fires with a shortened RR-interval (tᵢ₋₁), returning cycle would be prolonged if the sinoatrial node (SA node) is not electrically penetrated by the electrical wave of PAC. The electrical wavefronts of PAC and the SA node collide somewhere in the atrium, and the returning cycle would compensate the short coupling interval, and the summation of coupling interval and returning cycle would equal to two times of sinus cycle length. Even when a PAC with a further shorter RR-interval is encounter, the electrical wavefront would penetrate and reset the SA, node and the return cycle would be nearly the same as basic sinus cycle length. Since the PAC falls in the last 60˜80% of the basic sinus cycle would fall in the above conditions, the length would not be random and would depend on the characteristics of SA node and PAC coupling interval (tᵢ₋₁) This condition remains similar for PVC since its electrical wave must penetrate the atrioventricular node (AV node) first and requires a longer conduction time before reaching the SA node.

We suggested that coupling interval (tᵢ₋₁) and returning cycle (tᵢ) would present a stable relationship in patients with PAC or PVC. Since the flux (Hᵢ) is associated with tᵢ₋₁ as explained in the hemodynamic section, and the coupling interval is presented with constant more frequently, the flux-interval plot of returning cycle (tᵢ, Hᵢ) would be non-random and present with a few clusters of points. On the contrary, although the flux-interval plot of (tᵢ₋₁, Hᵢ) presents with a positive linear slope in AFib, the flux-interval plot of (tᵢ, Hᵢ) would be dispersed because of the low correlation of tᵢ₋₁ and tᵢ is low in AFib.

Therefore, in an embodiment, the atrial fibrillation detection system 10 of the present invention detects atrial fibrillation by calculating the III index 300 and RMSE of Main Cluster 400 for each signal 252 and determines that atrial fibrillation exists in a signal 252 if the III index 300 is above a III threshold value 302 and/or the RMSE of Main Cluster 400 is above a RMSE threshold value 402. In an embodiment, the processor 222 is configured to calculate the III 300 and RMSE of Main Cluster 400 for a signal segment 252. In an embodiment, the processor 222 is configured to determine that atrial fibrillation exists in a signal 252 if the III index 300 is above a III index threshold value 302 and/or the RMSE of Main Cluster 400 is above a RMSE threshold value 402.

In an embodiment, the III threshold value 302 is about 5 to about 70, such as about 5, about 10, about 15, about 20, about 25, about 30, about 35, about 40, about 45, about 50, about 55, about 60, about 65 or about 70 including any numbers or number ranges falling within these values. In an embodiment, the RMSE threshold value 402 is about 0.01 to about 0.2 such as about 0.01, about 0.02, about 0.03, about 0.04, about 0.05, about 0.06, about 0.07, about 0.08, about 0.09, about 0.1, about 0.11 about 0.12, about 0.13, about 0.14, about 0.15, about 0.16, about 0.17, about 0.18, about 0.19 or about 0.2 including any numbers or number ranges falling within these values.

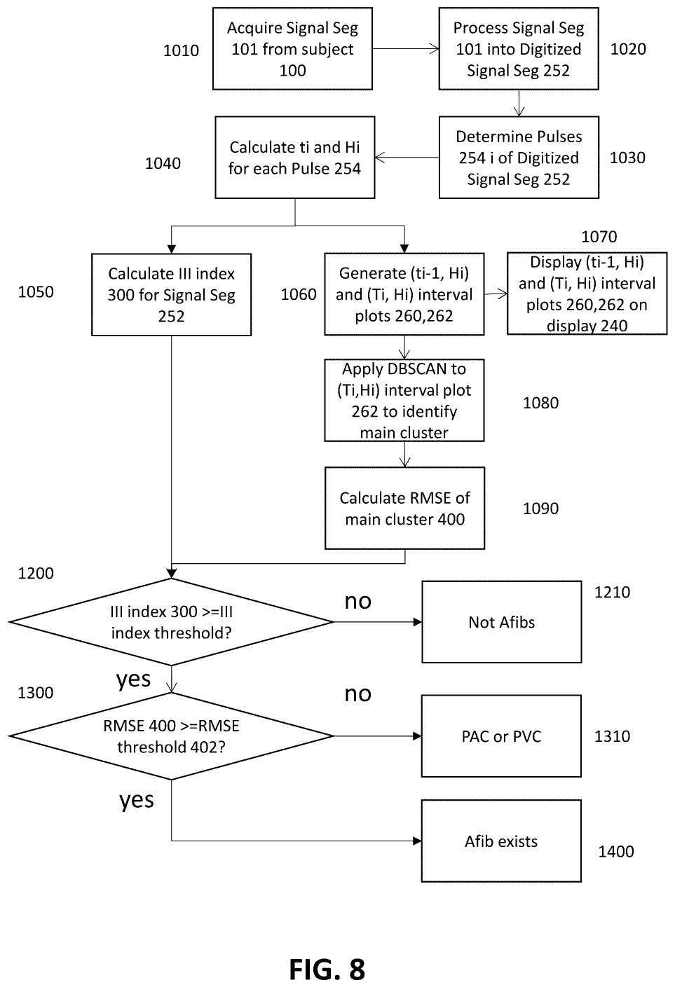

The present invention also provides a method of atrial fibrillation detection. FIG. 8 illustrates an embodiment of the atrial fibrillation detection method 1000 of the present invention. The method of the present invention comprises step 1010 of acquiring signal segment 101 from subject 100. In an embodiment, step 1010 comprises the subject 100 wearing sensor 114 using controller 200 to acquire signal segment 101 using sensor 114. Next in step 1020, the A/D converter 220 converts signal segment 101 into a digitized signal segment 252 and stores it in memory 250. In step 1030, the processor 222 determines individual pulses i of the digitized signal segment 252. Such individual pulse determination may include peak and valley detection of pulses. Next, the processor 222 calculates tᵢ 256 and Hᵢ 258 for each pulse 254 in step 1040 and stores this information in memory 250. In step 1050, the processor 222 calculates the III index 300 for a digitized signal segment 252. Next, in step 1060, the processor 222 generates flux-interval plots (tᵢ, Hᵢ) 260 and (tᵢ₋₁, Hᵢ) 262. The flux-interval plots 260, 262 may be displayed on display 240 in step 1070 for visual confirmation by the subject 100. The plots 260, 262 provide transparency in the form of visualization of the signal processing of the present invention. Such visualization allows the subject 100 to peer into some of the logic of the present invention and confirm the veracity of the resulting diagnosis of the present invention.

In step 1080, the processor 222 identifies the main cluster within the (tᵢ, Hᵢ) flux-interval plot 260. In an embodiment the processor 222 applies DBSCAN to the flux-interval plot 260 to identify the main cluster. After identifying the main cluster, the processor 222 calculates the RMSE of main cluster 400 in step 1080.

In step 1200, the processor 222 determines whether the III index 300 is greater than or equal to the index threshold 302. If the III index 300 is not greater or equal to the index threshold 302, then the processor 222 determines in step 1210 that no AFib exists in the signal segment. If the III index 300 is greater or equal to the index threshold 302, in step 1300, the processor 222 determines whether the RMSE of main cluster 400 is greater or equal to the RMSE threshold 402. If the RMSE of main cluster 400 is not greater or equal to the RMSE threshold 402, then in step 1310 the processor 222 labels the signal segment 101 as PAC or PVC. If the RMSE of main cluster 400 is greater or equal to the RMSE threshold 402, in step 1400 the processor 222 labels the signal segment 101 as containing atrial fibrillation. The result is displayed to the user using display 240. The display 240 may also concurrently display the flux-interval plots 260, 262 to provide visual confirmation to the subject 100, providing transparency to the subject 100.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Material and Methods

In this study, 5264 samples of 1-minute ECG and PPG signal segments 101 were recorded from 2632 subjects. The study was started by recruiting patient in community-based health care condition and aimed to exploit the potential utilization of PPG in blood chemistry test and other physiology signal on general population. All subjects were fully informed and have given written consent for the recording and usage of data in this study. Samples with AFib are labeled from ECG as a reference to PPG signal segments 101. The study was approved by the Institutional Review Board of Academia Sinica, Taiwan (Application No: AS-IRB01-16081).

### Measurement Protocol

The test subjects are asked to sit on the chair for rest condition in at least 5 minutes for a questionnaire. Personal information with sex, age, smoking habit, familial history of disorders, height, weight, waist circumference, SpO2 (peripheral oxygen saturation), blood pressure, blood glucose, HbA1C, are asked or measured by commercial products listed in the next section. The subjects are then set up with ECG patches for lead I angle and PPG finger clips on index fingers for consecutive two 1-minute recordings of waveform signals.

### Hardware

The devices and instruments for the experiment are as follows: digiO2 POM-201 for SpO2. Omron HEM-7320 for blood pressure. Roche Accu-check mobile for blood glucose. SEIMENS DCA Vantage Analyzer for HbA1C. CardioChek PA analyzer for blood lipid. The signal of PPG is recorded from TI (Texas Instruments) AFE4490 module and ECG from ADI (Analog Devices Inc.) AD8232-EVALZ.

### Data Preprocessing

After basic filtering of 0.1˜40 Hz, automated valley and peak detection on PPG signal segments 252 were conducted. Python 3 modules peakutils (ver. 1.1.1) were applied for the valley and peak detection. Peak detection was applied and cross-validated with corresponding two neighboring valleys. Valleys were also validated with continuous positive slopes. Based on the valleys, the PPG signal segments 252 is broken into singular pulses for later use.

Sample's cardiac rhythm was labeled based on its ECG signals accordingly by corresponding author Y. T. Chang, who serves as a cardiology specialists and clinical electrophysiologist since 2017. The labeled samples were selected from all samples but with interval irregularity index 300 (III, see Method section) above 10. In addition, we randomly selected 245 samples with III 300 under 10 to balance the distribution and to approximate the 10:1 ratio of non-AFib vs AFib. The results of labeled as ground truth are 286 NSR, 73 PAC (premature atrial contraction), 59 PVC (premature ventricle contraction), 40 AFib, and 2 atrioventricular block attributed to other type of arrhythmic rhythms.

### Method

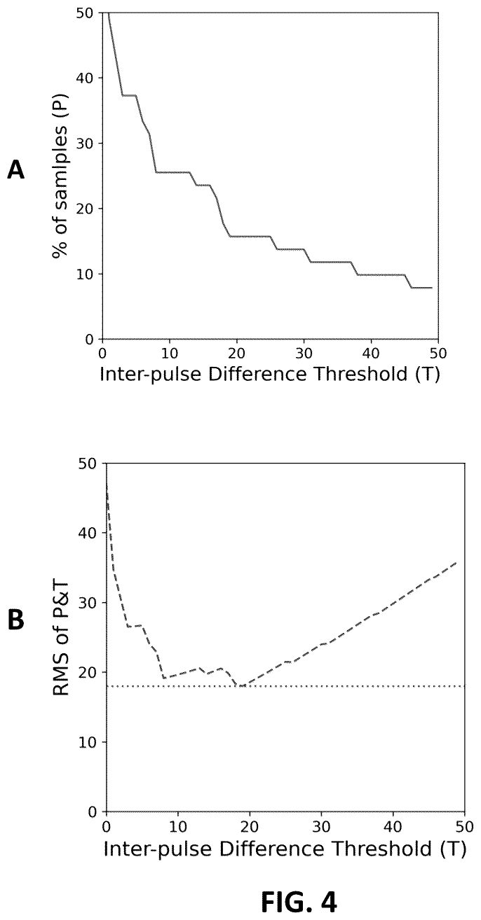

First, an H-index inspired index is designed to represent the irregularity of each sample set and be used to determine if the signal segment contains enough possible AFib pulses. The index is calculated by finding the smallest quadratic mean (root mean square) of the thresholds of percentage difference of consecutive pulse interval (T) and the proportion of pulse satisfied said interval threshold (P) as summarized in Equation (1). While previous studies often use RR time interval features in an absolute time difference of millisecond we opt to use the relative difference in % when it comes to inter-pulse difference. We believe the change in pulse length should take the specific person's current heart rate into account. A 10-millisecond change in pulse between a person with a heart rate of 60 beats/minute and another person with a heart rate of 80 beats/minute do have a significant difference. Thus, we use the relative difference in percentage instead of using an absolute value shared across different samples. This resulting value will be the interval irregularity index for the minute-long signal sample and be denoted as III 300. The process of finding the III 300 is illustrated in FIG. 4.

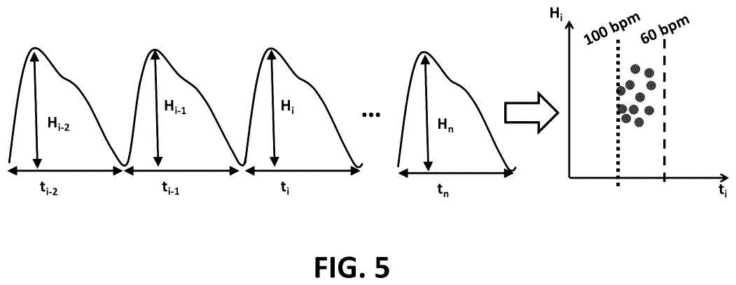

Then, for each pulse segmented by valley detection, the sequences of pulse amplitudes (Hᵢ) were normalized by dividing individual pulse peak amplitude with its average then paired together with intervals with different offsets. For each sample, the sets of (tᵢ₋₁, Hᵢ) and (tᵢ, Hᵢ) were then visualized into a flux-interval plots 260, 262 as shown in FIG. 5 to form the basis of our method. From the flux-interval plot 260, we can observe that NSR, PAC, PVC, and AFib samples display distinctively different patterns. Flux-interval plots 260, 262 allow user to assess their changes in cardio-output over time and identify different cardiac rhythms with their distinctive patterns much like identifying AFib from ECG using only a few iconic criteria. We expect any well-informed user can readily differentiate normal and abnormal rhythms easily based on the unique patterns without needing much training.

On each sample set of (tᵢ₋₁, Hᵢ) and (tᵢ, Hᵢ), DBSCAN (Density-based spatial clustering of applications with noise) clustering was applied. Conceptually the clustering result would reflect whether the types of pulses are within the signal based on each pulse's location flux-interval plot. The orthogonal regression was then applied on the main cluster (the cluster with the most data) of (tᵢ, Hᵢ) sets and the residual errors were recorded in the form of root-mean-squared error (RMSE). As shown in the examples of FIG. 6, we expect that the fitted regression line of the main cluster would result in a smaller RMSE on premature contractions as they tend to have tight clusters as opposed to the more scattered distribution of AFib on the flux-interval plot. We intend to use this, denoted “Regression RMSE of Main Cluster”, as a simple index to represent the degree of dispersion of each sample.

### Results

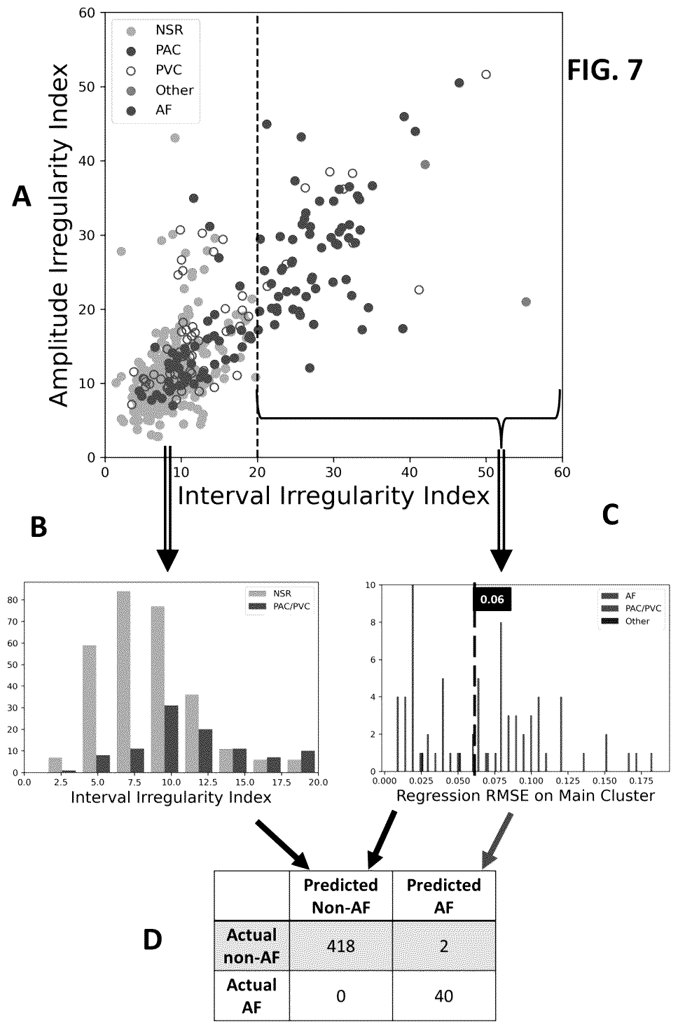

Based on our data set, we found that with two simple conditions of checking irregularity and the regression RMSE of the main cluster, we can correctly determine if the 60 seconds PPG signal segments 252 contain AFib with only two false-positives without any false-negative. In FIG. 7, we visualized how each condition separates different sinus rhythms in a tree-like structure. The same steps of acquiring interval irregularity were adopted on amplitude here for the purpose of matching the scale of the X and Y axis. From the top plot FIG. 7A, we can clearly see AFib samples (red dots) have significantly larger irregularities compared to the others and with some premature contractions data in the mix. With an interval irregularity of 20 as the boundary, all NSR and AFib could be separated perfectly. Despite being labeled as premature contractions, the data with irregularity under 20 are generally NSR with occasional premature contractions which are considered normal and mostly harmless. On the other hand, concerning the premature contraction data with irregularity over 20, these PAC and PVC usually have a bigeminy, trigeminy, or even quadrigeminy rhythm which leads to two or more distinctive clusters each with tight distribution on their flux-interval plot. Based on the features previous studies used, differentiating these PAC and PVC with large irregularity from AFib is what their methods may struggle on. In FIG. 7C, we can see that by assessing the orthogonal regression RMSE of main cluster we can convincingly pick out the AFib data from the pile of data that had irregularity over 20 with regression RMSE of main cluster at 0.06 as the boundary. Based on these two simple yet powerful threshold conditions that require no complex computation we can automate the classification of AFib with the performance of 1, 0.995, 0.995, and 0.952 for sensitivity, specificity, accuracy, and precision, respectively. This result suggests our model outperformed the majority of the 24 studies reviewed by Pereira et al.

FIGS. 9A and 9B demonstrated how the (tᵢ₋₁, Hᵢ), (tᵢ, Hᵢ) relations generally manifest themselves for different types of cardiac rhythms on the flux-interval plot. The most iconic and representative characteristics are summarized in Table 1 as a guideline for differentiating types of cardiac rhythms.

TABLE 1
Distinct flux-interval plot characteristics for visual reassessment.

| | Hᵢ vs tᵢ₋₁ | Hᵢ vs tᵢ | Data distribution |
|---|---|---|---|
| AFib | Positive linear | Pulses interval often | Single cluster with wide |
| | correlation | exceed 0.6 s (100 bpm) | variation on both flux and |
| | Pulses interval often | | interval |
| | exceed 0.6 s (100 bpm) | | |
| PAC/PVC | Typically with more | Typically with more | low variation within |
| | vertical pattern and | vertical pattern and | individual cluster |
| | multiple clusters | multiple clusters | |
| NSR | Compact pattern | Compact pattern | Single cluster with small |
| | | | variation on interval |

NSR would mostly be in a single tight cluster with little variation both in terms of interval and amplitude. PAC and PVC with rhythmic premature contraction would often present themselves as two or more distinct cluster of premature beats, normal beats, and the extended normal beat right after each premature beat. For samples of NSR, PAC, and PVC, their style of data distribution would appear consistent on both (tᵢ₋₁, Hᵢ) 262 and (tᵢ, Hᵢ) 260 flux-interval plots. On the other hand, the AFib pattern would often look like a semi-tight scatter of points forming a positive slope on the (tᵢ₋₁, Hᵢ) plot 262 and a widely scattered distribution on the (tᵢ, Hᵢ) plot 260.

This visualization helps us understand the fundamental difference between types of cardiac rhythms for classification. The observed distinct characteristic of different rhythms on the flux- interval plot is consistent with our hypothesis on identifying cardiac rhythms based on similar characteristic when identifying them via ECG. The plots cluster pattern correlates to the functionality of the atrium and the irregularity reflects the rhythmicity. Though with the cluster pattern we can differentiate AFib from PAC and PVC, however it appears that PAC and PVC are in-distinguishable on the flux-interval plot.

### Discussion

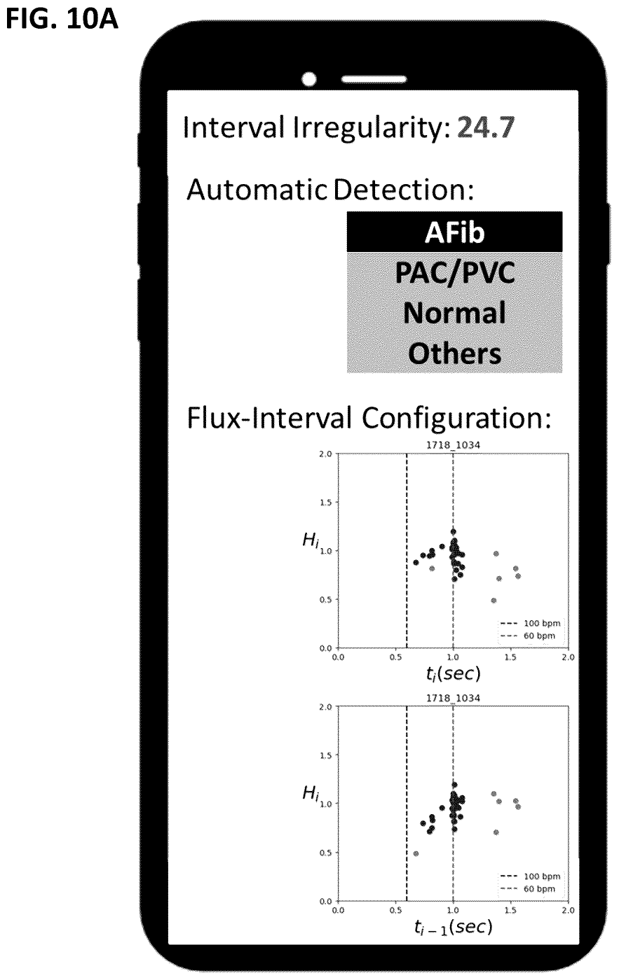

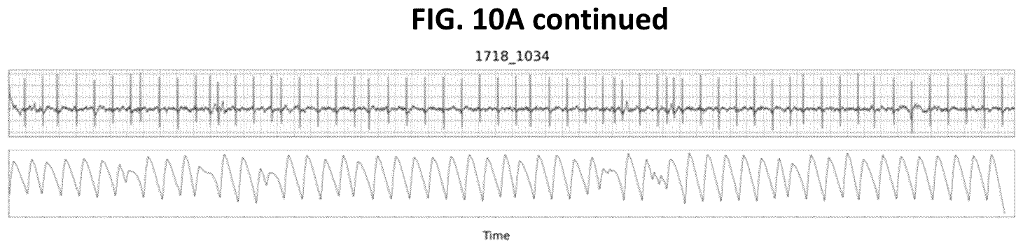

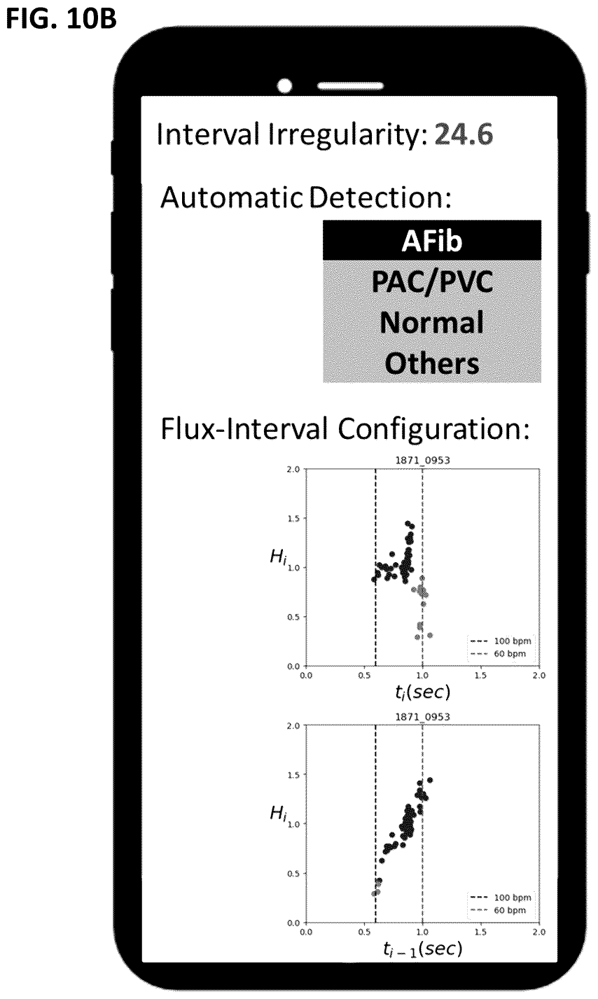

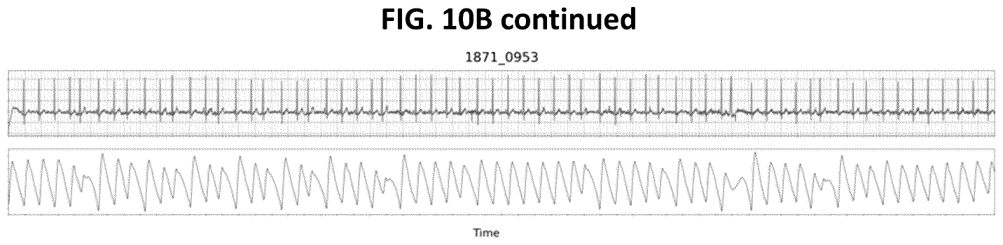

Compared to previous studies, our approach is more in line with the statistical analysis with assistance from machine learning methods while focusing heavily on deriving meaningful physiological features via novel approaches. While many previous work have already achieved perfect or near perfect results through different algorithms, their behind the scene processes are hidden to the users like a black box. With our innovative approach with the flux-interval plot, we can provide a visual presentation on why and how we can effectively and convincingly distinguish AFib from other types of cardiac arrhythmia. While introducing a novel predictive analytic model to new users, their question on its usability often resides in how accurate is it and how can they trust the result. Our straightforward model offers good accuracy on par with the best performing studies while added intuitive visual presentation allows the user to carry out a “trust, but verify” approach. The extra information grants users access to a more informative result and the ability to double-check the basis of each prediction with their own eyes for increasing use case confidence. This has the potential to bring significant improvement and contribute to new functionality to the PPG-based AFib detection method in the future. While our method produces accurate automated prediction via simple parameters, there still are 2 false-positive cases of non-AFib arrhythmia classified as AFib as presented in FIG. 10, which we will address below.

### Visual Reassessment

FIGS. 10A and 10B present two false-positive cases of ECG labeled PAC samples misclassified as AFib by our automated detection while their flux-interval plot tells a different story. Upon visually reassessing the flux-interval plots in both configurations following the guideline in Table 1, we can clearly see that in both cases the (tᵢ, Hᵢ) plot 260 did not present itself in a single widely scattered cluster and multiple clusters can be identified. In the case of FIG. 10A, it appears as sinus rhythms with PAC but with a short episode of atrial tachycardia (multiple APC in short succession) in the mix at around the 39 second marks, thus resulted in the resemblance of AFib. In the case of FIG. 10B, the false positive lies in the presence of various kinds of PAC with different coupling interval (tᵢ₋₁). These false positive results could be attributed to the limitation of the clustering algorithm to differentiate these turbulences of RR-interval when complex arrhythmias happen. Even when reading ECG signal, these complex arrhythmias are frequently confusing and require clear ECG P-wave to figure out the answer in clinical practice.

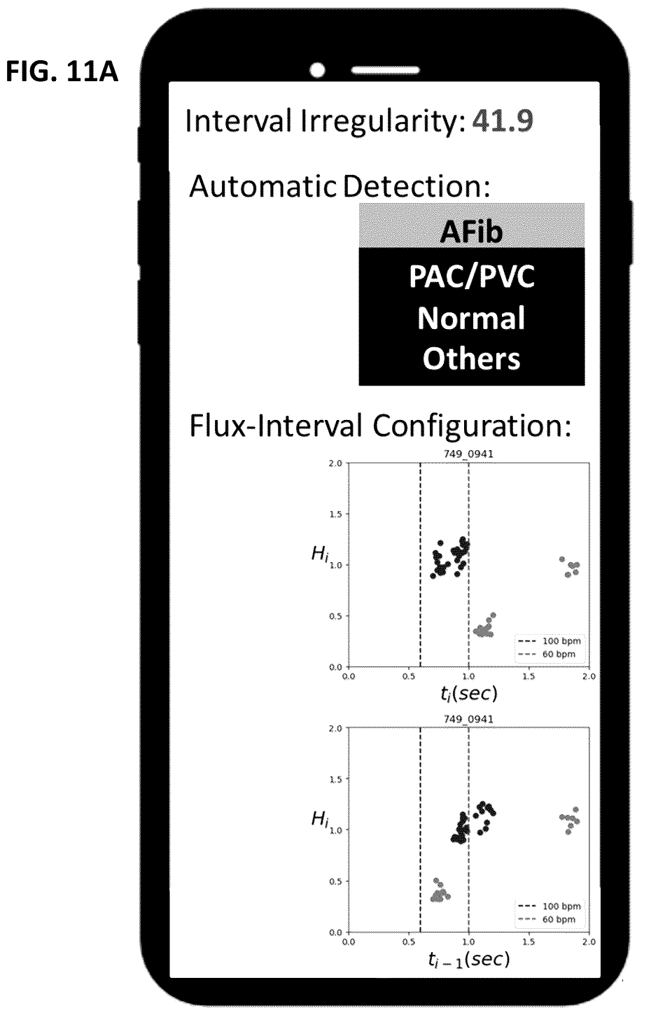

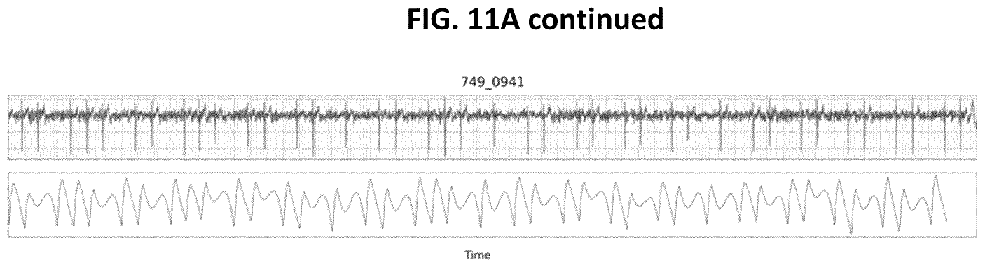

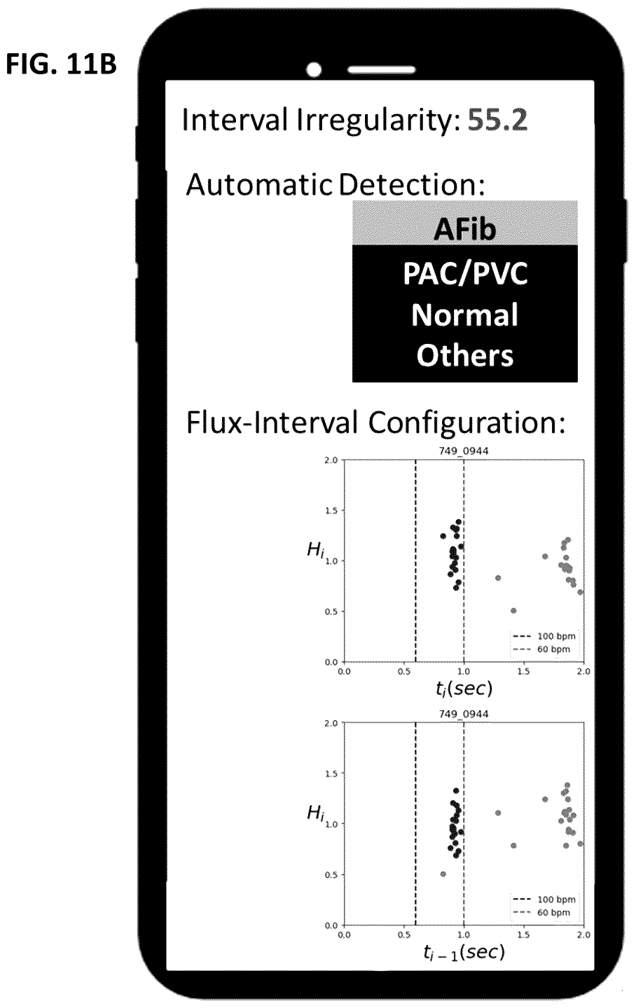

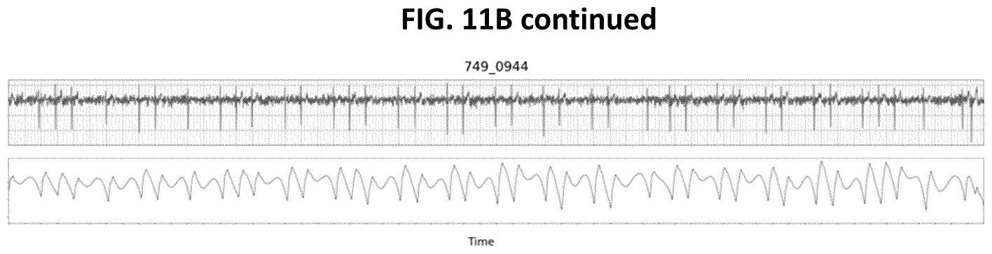

In FIGS. 11A and 11B we show the two cases of atrioventricular block labeled as others, which are automatically detected as non-AFib. In both cases, their irregularity indexes are very high but their regression RMSE on main cluster is less than the threshold of 0.06. Thus, they are correctly detected as non-AFib. From looking their flux-interval configurations following the guideline in Table 1, they are very similar to PAC/PVC but far from AFib. This suggests that even other types of arrhythmias may have the similar characteristics with PAC/PVC on the flux-interval plots and can too be easily differentiated from AFib. The two cases demonstrated reassessment with flux-interval configuration is very useful for users to confirm automatic classification result.

### Conclusion

PPG signals provided blood flux information previously unavailable through ECG had shed new light on new methods for detecting atrial fibrillation and other arrhythmia patterns. In this study, we demonstrated how monitoring one's change in blood flux (represented by PPG pulse amplitude) across a period of time can be an easy and effective way of detecting if the subject is having an atrial fibrillation episode. We found when projecting the PPG waveform data to the flux-interval plots 260. 262, based on explainable physiology relation between pulse interval and amplitude, AFib could be easily characterized and convincingly differentiated from other arrhythmic rhythms. Previous AFib detection usually suffering from conjunction with PAC/PVC by PPG if only counting on interval randomness has been solved either through the automatic detection process or visual reassessment with this novel technology. While our proposed PPG method offers significant benefits over previous methods, like any other PPG based method, it is still limited by the quality of PPG signal. The proposed method with automated detection on AFib shows a combined sensitivity, specificity, accuracy, and precision of 1, 0.995, 0.995, and 0.952 across 460 samples studied. Due to the small sample size of this study, studies with a larger sample size and on population with some common types of heart disease could further validate the robustness and applicability of the methodology.

### REFERENCES

Each reference is hereby incorporated in each of its entirety.
1. Lee, E.; Choi, E. K.; Han, K. D.; Lee, H.; Choe, W. S.; Lee, S. R.; Cha, M. J.; Lim, W. H.; Kim, Y. J.; Oh, S. Mortality and causes of death in patients with atrial fibrillation: A nationwide population-based study. PLOS One 2018, 13, e0209687, doi: 10.1371/journal.pone.0209687.
2. Hindricks, G.; Potpara, T.; Dagres, N.; Arbelo, E.; Bax, J. J.; Blomstrom-Lundqvist, C.; Boriani, G.; Castella, M.; Dan, G. A.; Dilaveris, P. E.; et al. 2020 ESC Guidelines for the diagnosis and management of atrial fibrillation developed in collaboration with the European Association for Cardio-Thoracic Surgery (EACTS): The Task Force for the diagnosis and management of atrial fibrillation of the European Society of Cardiology (ESC) Developed with the special contribution of the European Heart Rhythm Association (EHRA) of the ESC. Eur Heart J 2021, 42, 373-498, doi:10.1093/eurheartj/ehaa612.
3. Eerikainen, L. M.; Bonomi, A. G.; Schipper, F.; Dekker, L. R. C.; Vullings, R.; de Morree, H. M.; Aarts, R. M. Comparison between electrocardiogram-and photoplethysmogram-derived features for atrial fibrillation detection in free-living conditions. Physiol Meas 2018, 39, 084001, doi: 10.1088/1361-6579/aad2c0.
4. Paneni, F.; Beckman, J. A.; Creager, M. A.; Cosentino, F. Diabetes and vascular disease: pathophysiology, clinical consequences, and medical therapy: part I. Eur Heart J 2013, 34, 2436-2443, doi:10.1093/eurheartj/eht149.
5. Benichou, T.; Pereira, B.; Mermillod, M.; Tauveron, I.; Pfabigan, D.; Maqdasy, S.; Dutheil, F. Heart rate variability in type 2 diabetes mellitus: A systematic review and meta-analysis. PLOS One 2018, 13, e0195166, doi:10.1371/journal.pone.0195166.
6. Alian, A. A.; Shelley, K. H. Photoplethysmography. Best Pract Res Clin Anaesthesiol 2014, 28, 395-406, doi:10.1016/j.bpa.2014.08.006.
7. Bashar, S. K.; Han, D.; Hajeb-Mohammadalipour, S.; Ding, E.; Whitcomb, C.; McManus, D. D.; Chon, K. H. Atrial Fibrillation Detection from Wrist Photoplethysmography Signals Using Smartwatches. Sci Rep 2019, 9, 15054, doi:10.1038/s41598-019-49092-2.
8. Pereira, T.; Tran, N.; Gadhoumi, K.; Pelter, M. M.; Do, D. H.; Lee, R. J.; Colorado, R.; Meisel, K.; Hu, X. Photoplethysmography based atrial fibrillation detection: a review. NPJ Digit Med 2020, 3,3, doi: 10.1038/s41746-019-0207-9.
9. Lee, J.; Nam, Y.; McManus, D. D.; Chon, K. H. Time-varying coherence function for atrial fibrillation detection. IEEE Trans Biomed Eng 2013, 60, 2783-2793, doi:10.1109/TBME.2013.2264721.
10. Bonomi, A. G.; Schipper, F.; Eerikäinen, L. M.; Margarito, J.; Aarts, R. M.; Babaeizadeh, S.; Morree, H. M. d.; Dekker, L. Atrial fibrillation detection using photo-plethysmography and acceleration data at the wrist. In Proceedings of the 2016 Computing in Cardiology Conference (CinC), 11-14 Sep. 2016, 2016; pp. 277-280.
11. Mc, M. D.; Chong, J. W.; Soni, A.; Saczynski, J. S.; Esa, N.; Napolitano, C.; Darling, C. E.; Boyer, E.; Rosen, R. K.; Floyd, K. C.; et al. PULSE-SMART: Pulse-Based Arrhythmia Discrimination Using a Novel Smartphone Application. J Cardiovasc Electrophysiol 2016, 27, 51-57, doi:10.1111/jce.12842.
12. Conroy, T.; Guzman, J. H.; Hall, B.; Tsouri, G.; Couderc, J. P. Detection of atrial fibrillation using an earlobe photoplethysmographic sensor. Physiol Meas 2017, 38, 1906-1918, doi:10.1088/1361-6579/aa8830.
13. Eckstein, J.; Krivoshei, L.; Weber, S.; Burkard, T.; Maseli, A.; Brasier, N.; Kuhne, M.; Conen, D.; Huebner, T.; Seeck, A. Smart detection of atrial fibrillationdagger. Europace 2017, 19, 753-757, doi:10.1093/europace/euw125.
14. O′Shea, K.; Nash, R. An Introduction to Convolutional Neural Networks. ArXiv e-prints 2015.
15. Kwon, S.; Hong, J.; Choi, E. K.; Lee, E.; Hostallero, D. E.; Kang, W. J.; Lee, B.; Jeong, E. R.; Koo, B. K.; Oh, S.; et al. Deep Learning Approaches to Detect Atrial Fibrillation Using Photoplethysmographic Signals: Algorithms Development Study. JMIR Mhealth Uhealth 2019, 7, e12770, doi:10.2196/12770.
16. Tarniceriu, A.; Harju, J.; Yousefi, Z. R.; Vehkaoja, A.; Parak, J.; Yli-Hankala, A.; Korhonen, I. The Accuracy of Atrial Fibrillation Detection from Wrist Photoplethysmography. A Study on Post-Operative Patients. Annu Int Conf IEEE Eng Med Biol Soc 2018, 2018, 1-4, doi:10.1109/EMBC.2018.8513197.
17. Lemay, M.; Fallet, S.; Renevey, P.; Solà, J.; Leupi, C.; Pruvot, E.; Vesin, J. Wrist- located optical device for atrial fibrillation screening: A clinical study on twenty patients. In Proceedings of the 2016 Computing in Cardiology Conference (CinC), 11-14 Sep. 2016, 2016; pp. 681-684.
18. Tison, G. H.; Sanchez, J. M.; Ballinger, B.; Singh, A.; Olgin, J. E.; Pletcher, M. J.; Vittinghoff, E.; Lee, E. S.; Fan, S. M.; Gladstone, R. A.; et al. Passive Detection of Atrial Fibrillation Using a Commercially Available Smartwatch. JAMA Cardiology 2018, 3, 409-416, doi:10.1001/jamacardio.2018.0136.
19. Nemati, S.; Ghassemi, M. M.; Ambai, V.; Isakadze, N.; Levantsevych, O.; Shah, A.; Clifford, G. D. Monitoring and detecting atrial fibrillation using wearable technology. Annu Int Conf IEEE Eng Med Biol Soc 2016, 2016, 3394-3397, doi:10.1109/EMBC.2016.7591456.
20. Tang, S.C.; Huang, P. W.; Hung, C. S.; Shan, S. M.; Lin, Y. H.; Shieh, J. S.; Lai, D. M.; Wu, A. Y.; Jeng, J. S. Identification of Atrial Fibrillation by Quantitative Analyses of Fingertip Photoplethysmogram. Sci Rep 2017, 7, 45644, doi:10.1038/srep45644.
21. Bashar, S. K.; Han, D.; Soni, A.; McManus, D. D.; Chon, K. H. Developing a novel noise artifact detection algorithm for smartphone PPG signals: Preliminary results. In Proceedings of the 2018 IEEE EMBS International Conference on Biomedical & Health Informatics (BHI), 4-7 Mar. 2018, 2018; pp. 79-82.
22. Chong, J. W.; Cho, C. H.; Tabei, F.; Le-Anh, D.; Esa, N.; Mcmanus, D. D.; Chon, K. H. Motion and Noise Artifact-Resilient Atrial Fibrillation Detection Using a Smartphone. IEEE Journal on Emerging and Selected Topics in Circuits and Systems 2018, 8, 230-239, doi:10.1109/JETCAS.2018.2818185.
23. Shan, S.- M.; Tang, S.- C.; Huang, P.- W.; Lin, Y.- M.; Huang, W.- H.; Lai, D.- M.; Wu, A.- Y. A. Reliable PPG-based algorithm in atrial fibrillation detection. In Proceedings of the 2016 IEEE Biomedical Circuits and Systems Conference (BioCAS), 2016; pp. 340-343.
24. Corino, V. D. A.; Laureanti, R.; Ferranti, L.; Scarpini, G.; Lombardi, F.; Mainardi, L. T. Detection of atrial fibrillation episodes using a wristband device. Physiol Meas 2017, 38, 787-799, doi:10.1088/1361-6579/aa5dd7.
25. Fallet, S.; Lemay, M.; Renevey, P.; Leupi, C.; Pruvot, E.; Vesin, J. M. Can one detect atrial fibrillation using a wrist-type photoplethysmographic device? Med Biol Eng Comput 2019, 57, 477-487, doi:10.1007/s11517-018-1886-0.
26. Aliamiri, A.; Shen, Y. Deep learning based atrial fibrillation detection using wearable photoplethysmography sensor. In Proceedings of the 2018 IEEE EMBS International Conference on Biomedical & Health Informatics (BHI), 4-7 Mar. 2018, 2018; pp. 442-445.
27. Poh, M. Z.; Poh, Y. C.; Chan, P. H.; Wong, C. K.; Pun, L.; Leung, W. W.; Wong, Y. F.; Wong, M. M.; Chu, D. W.; Siu, C. W. Diagnostic assessment of a deep learning system for detecting atrial fibrillation in pulse waveforms. Heart 2018, 104, 1921-1928, doi:10.1136/heartjnl-2018-313147.
28. Gotlibovych, I.; Crawford, S.; Goyal, D.; Liu, J.; Kerem, Y.; Benaron, D.; Yilmaz, D.; Marcus, G.; Li, Y. End-to-end deep learning from raw sensor data: Atrial fibrillation detection using wearables. arXiv preprint arXiv: 1807.10707 2018.
29. Voisin, M.; Shen, Y.; Aliamiri, A.; Avati, A.; Hannun, A.; Ng, A. Ambulatory atrial fibrillation monitoring using wearable photoplethysmography with deep learning. arXiv preprint arXiv: 1811.07774 2018.
30 Shashikumar, S. P.; Shah, A. J.; Clifford, G. D.; Nemati, S. Detection of paroxysmal atrial fibrillation using attention-based bidirectional recurrent neural networks. In Proceedings of the Proceedings of the 24th ACM SIGKDD International Conference on Knowledge Discovery & Data Mining, 2018; pp. 715-723.
31. Shashikumar, S. P.; Shah, A. J.; Li, Q.; Clifford, G. D.; Nemati, S. A deep learning approach to monitoring and detecting atrial fibrillation using wearable technology. In Proceedings of the 2017 IEEE EMBS International Conference on Biomedical & Health Informatics (BHI), 2017; pp. 141-144.
32. Schack, T.; Harb, Y. S.; Muma, M.; Zoubir, A. M. Computationally efficient algorithm for photoplethysmography-based atrial fibrillation detection using smartphones. In Proceedings of the 2017 39th Annual International Conference of the IEEE Engineering in Medicine and Biology Society (EMBC), 11-15 Jul. 2017, 2017; pp. 104-108.
33. Pfeiffer, K. P.; Kenner, T.; Schaefer, J. Application of statistical methods for the analysis or interval related cardiac performance variations during cardiac arrhythmia in man. Cardiovasc Res 1984, 18, 80-98, doi:10.1093/cvr/18.2.80.
34. Bootsma, B. K.; Hoelsen, A. J.; Strackee, J.; Meijler, F. L. Analysis of R-R intervals in patients with atrial fibrillation at rest and during exercise. Circulation 1970, 41, 783-794, doi:10.1161/01.cir.41.5.783.
35. Maruyama, T.; Fukata, M. Increased coupling interval variability--mechanistic, diagnostic and prognostic implication of premature ventricular contractions and underlying heart diseases. Circ J 2015, 79, 2317-2319, doi:10.1253/circj.CJ-15-0963.
36. Callans, D. J. Josephson's Clinical Cardiac Electrophysiology: Techniques and Interpretations, 6th ed.; Lippincott Williams & Williams: Philadelphia, 2020.

What is claimed is:

1. An atrial fibrillation detection system comprising:
a signal sensor configured to read a cardiovascular signal segment from a subject;
a processor configured to receive the signal segment from the signal sensor and perform signal processing on the signal segment;
wherein the processor identifies individual pulses within the signal segment;
wherein the processor calculates pulse interval time ti for one or more of the individual pulses where the pulse interval time ti is the time duration of the i^{th} individual pulse;
wherein the processor determines irregularity in pulse interval time ti of the signal segment based on the percentage difference in pulse interval times of consecutive pulses; and
wherein the processor determines existence of atrial fibrillation in the signal segment if the irregularity in the pulse interval time of the signal segment exceeds interval time irregularity threshold.

2. The system of claim 1, wherein the irregularity in pulse interval time comprises interval irregularity index (III) calculated according to equation (1)
$$III = min {\{ \sqrt{\frac{{T}_{j}^{2} + {P}_{j}^{2}}{2}} \}}_{j = 1}^{N}$$
wherein III is the interval irregularity index, Tⱼ is inter-pulse percentage difference threshold and has value of j % and Pⱼ is the proportion of pulses in the signal segment whose percentage interval time difference of consecutive pulse intervals is equal to or greater than the inter-pulse difference threshold Tⱼ.

3. The system of claim 2, wherein the processor determines existence of atrial fibrillation if the interval irregularity index III of the signal segment is equal to or greater than interval irregularity index threshold.

4. The system of claim 3, wherein the interval irregularity index threshold is about 5 to about 40.

5. The system of claim 1, wherein the processor screens out any false positives due to non-atrial fibrillation contractions in the signal segment such as premature atrial contraction (PAC) and/or premature ventricular contraction (PVC) using a false positive screening process.

6. The system of claim 5, wherein the false positive screening process is based on orthogonal regression on the main cluster wherein the main cluster is the cluster with the most data in flux-interval plot (tᵢ, Hᵢ) where Hᵢ is the pulse interval normalized amplitude calculated as peak amplitude of the i^{th} pulse divided by average amplitude of the same i^{th} pulse.

7. The system of claim 6, wherein the processor is configured to apply density-based spatial clustering of application with noise (DBSCAN) on the flux-interval plot (tᵢ, Hᵢ) to identify the main cluster.

8. The system of claim 7, wherein the false positive screening process comprises regression root mean squared error (RMSE) of main cluster calculated as the residual errors in the form of root-mean-squared error.

9. The system of claim 8, wherein the processor determines existence of atrial fibrillation if the RMSE of main cluster of the signal segment is equal to or greater than RMSE of main cluster threshold.

10. The system of claim 9, wherein the RMSE of main cluster threshold is about 0.01 to about 0.2.

11. The system of claim 1 further comprising a display configured to display flux-interval plots comprising a flux-interval plot of (tᵢ, Hᵢ) and a flux-interval plot of (tᵢ₋₁, Hᵢ) to allow the subject to visually reassess the atrial fibrillation determination based on the plot patterns wherein tᵢ is the duration of the i^{th} pulse and Hᵢ is the normalized amplitude of the i^{th} pulse.

12. The system of claim 11 wherein the subject may visually determine whether the flux-interval relations plots indicate atrial fibrillation if the flux-interval plot (tᵢ, Hᵢ) pattern presents a wide cluster with positive linear slope and/or if the flux-interval plot (tᵢ₋₁, Hᵢ) pattern presents a single widely scattered cluster;

13. The system of claim 1, wherein signal sensor comprises a photoplethysmogram (PPG) signal sensor.

14. A method of detecting atrial fibrillation comprising the steps of acquiring a cardiovascular signal segment from a subject using a signal sensor;
dividing the signal segment into a plurality of individual pulses using the processor such that pulse interval time ti is the time duration of the i^{th} pulse;
determining irregularity in the pulse interval time of the signal segment based on the percentage difference in the pulse interval times of consecutive pulses using the signal processor; and
determining existence of atrial fibrillation if the irregularity in the pulse interval time of the signal segment exceeds interval time irregularity threshold using the signal processor.

15. The method of claim 14, wherein the irregularity in interval time of the signal segment comprises interval irregularity index calculated according to equation (1)
$$III = min {\{ \sqrt{\frac{{T}_{j}^{2} + {P}_{j}^{2}}{2}} \}}_{j = 1}^{N}$$
wherein III is the interval irregularity index, Tⱼ is the inter-pulse difference threshold and has value of j % and Pⱼ is the proportion of pulses of the signal segment whose percentage interval time difference of consecutive pulses is larger than the corresponding inter-pulse difference threshold Tⱼ.

16. The method of claims 15, wherein the step of determining existence of atrial fibrillation in the signal segment comprises determining if the interval irregularity index III of the signal segment is equal to or greater than interval irregularity index threshold.

17. The method of claim 16, wherein the interval irregularity index threshold is about 5 to 40.

18. The method of claim 14, further comprising the step of screening out any false positives due to premature contractions such as PAC and/or PVC in the signal segment using a false positive screening process executed by the signal processor.

19. The method of claim 18, wherein the step of screening out any false positives is based on orthogonal regression on the main cluster wherein the main cluster is the cluster with the most data in flux-interval plot (tᵢ, Hᵢ) where Hᵢ is the normalized amplitude of the i^{th} pulse calculated by dividing peak amplitude of the i^{th} pulse by average amplitude of the same i^{th} pulse.

20. The method of claim 19, wherein the step of screening out any false positives comprises the step of applying density-based spatial clustering of application with noise (DBSCAN) on flux interval plot (tᵢ, Hᵢ) to identify the main cluster by the signal processor.

21. The method of claim 20, wherein the step of screening out any false positives further comprises the step of calculating regression root mean squared error (RMSE) of main cluster calculated as the residual errors in the form of root-mean-squared error.

22. The method of claims 21, wherein the step of screening out any false positives further comprises the step of determining existence of atrial fibrillation if the RMSE of main cluster of the signal is equal to or greater than RMSE of main cluster threshold.

23. The method of claim 22 wherein the RMSE of main cluster threshold is about 0.01 to about 0.2.

24. The method of claim 14 further comprising the step of displaying flux-interval relations plots comprising a flux-interval plot of (tᵢ, Hᵢ) and a flux-interval plot of (tᵢ₋₁, Hᵢ) on a display so that the subject is able to visually reassess the atrial fibrillation determination based on the plot pattern wherein tᵢ is the duration of the i^{th} interval and Hᵢ is the pulse interval normalized amplitude calculated as peak amplitude of the i^{th} pulse divided by average amplitude of the i^{th} pulse.

25. The method of claim 24 wherein the visual reassessment by the subject of the existence of atrial fibrillation in the signal segment comprises determining existence of atrial fibrillation if the (tᵢ, Hᵢ) flux interval plot pattern presents a wide cluster with positive linear slope and/or if the (tᵢ₋₁, Hᵢ) flux interval plot pattern presents a single widely scattered cluster.

26. The method of claim 14 wherein the step of reading the signal segment is performed using a PPG sensor and the rest of the steps are performed using a processor.

27. The method of claim 14 wherein cardiovascular signal segment comprises a PPG signal segment.
